(19) United States
(12) Patent Application Publication
(10) Pub. No.: US 2010/0156104 A1
(43) Pub. Date: Jun. 24, 2010

(54) Thermal Reduction Gasification Process for Generating Hydrogen and Electricity

(76) Inventor: N. Edward Bottinelli, Dallas, TX (US)

Correspondence Address: RALPH H. DOUGHERTY, 4219 KRONOS PLACE, CHARLOTTE, NC, 28210, US

(21) Appl. No.: 12/223,902

(22) PCT Filed: Mar. 23, 2006
(86) PCT No.: PCT/US2006/010694

§ 371 Date: Aug. 9, 2008

(51) Int. Cl. F01D 15/10; C10B 1/00; C25B 15/00; H01M 8/06; C12M 1/00; B01J 19/00; F02C 7/22

(52) U.S. Cl. 290/52; 48/89; 204/232; 429/422; 435/289.1; 422/189; 60/39.12

(57) ABSTRACT

An apparatus for generating synthesis gas from waste organic materials that consists of a thermal reduction gasification reactor which is a rotary reactor having a drying and volatilizing zone for gasifying organic materials and a reformation zone for converting the gasified organic materials to synthesis gas. Solid waste organic material is fed to the reactor that heats the solid material to a temperature of about 600° C. to about 1000° C. The synthesis gas generated by the apparatus is substantially hydrogen and carbon monoxide. The apparatus is combined with an electrical generation system for making purified hydrogen and electricity. Alternatively, the synthesis gas can be used as a source for hydrogen. The synthesis gas is cleaned, the composition is shifted to enrich the content of hydrogen, and the hydrogen is isolated from the other gases that make up the synthesis gas. Alternatively, the synthesis gas can be fermented forming an organic alcohol and an organic acid.

### BACKGROUND OF THE INVENTION

1) Field of the Invention

The invention relates generally to a method and apparatus for gasifying organic materials, and more particularly to a method and apparatus for generating molecular hydrogen from waste organic materials having fixed hydrogen, such as biomass, municipal solid waste, scrap tires, automobile shredder residue, and agricultural wastes.

2) Prior Art

It is known in the art that the thermal pyrolysis of coal can be used to produce petroleum-like distillates, which in the gaseous form are known as syncrude. In a similar process a gaseous fuel is formed from the partial oxidative combustion of natural gas forming a gaseous mixture of hydrogen and carbon monoxide. This gaseous mixture, which has excellent reducing properties, is commonly called synthesis gas or reform gas, and it is frequently employed in iron making and steel making to metallize iron oxide to iron at relatively low temperatures. Outside of iron making, reform gases are not often employed as a heat source as they have a lower heat content than natural gas. The heat of combustion of methane is 21528 BTU/Lb or 907 BTU/cu Ft. The heat of combustion of hydrogen is 51552 BTU/Lb or 273 BTU/cu Ft, and the heat of combustion of carbon monoxide is 4242 BTU/Lb or 330 BTU/cu Ft. On a volume basis, reform gas has about one third of the heat content of natural gas, however, on a weight basis, assuming there are equal molar percentages of carbon monoxide and hydrogen, the mixture has a heat of combustion of 7489 BTU/Lb. On a weight basis, hydrogen has a much higher heat content than natural gas. Hydrogen's only product of combustion is water and, therefore, has a very low environmental impact as a fuel. Because of its high heat of combustion and environmental friendliness, hydrogen has been identified as the fuel of choice to supplement or replace gasoline. Hydrogen is also reputedly less dangerous to handle than petroleum based fuels because it is so volatile that it will readily disperse if accidentally released, and the rate of dispersion is so fast as to minimize the possibility that there will be a sufficient quantity as to be present at an explosive level. In contrast, only a portion of gasoline is highly volatile.

A number of states, most notably California, have initiated studies to evaluate the feasibility of providing a hydrogen distribution network for cars and other vehicles powered by hydrogen or hybrid systems. The studies have generally settled on two feasible solutions, one where the generation facilities use electricity to generate hydrogen along the electrical power grid, and another where the hydrogen is generated centrally, and then distributed either as a compressed gas or a cryogenic liquid. The solutions recognize that most sources of energy are substantially concentrated, either as large generating facilities like hydroelectric, coal or nuclear plants or as refineries with tank farms. While there are economies of scale, large electrical generating facilities have significant losses in energy over the power grid, and the cost of the energy is further increased by the capital cost of the distribution network itself. Hydrogen distributed from a cracking facility such as a refinery has the added cost of distribution, either as a compressed gas or pipeline, and is dependent on oil.

What is needed is a system that can reliably generate a fuel that, either directly or indirectly, serves as a source for hydrogen, where the system would be substantially free standing, and capable of utilizing unconventional materials for power. The system preferably should require only a minimal distribution network and, where needed, be able to supplement an existing electrical grid.

### SUMMARY OF THE INVENTION

The invention provides a method and apparatus for thermally processing organic based raw materials of either primary or secondary origin in order to extract volatile organic vapors and to selectively produce non-condensable synthesis gases that are rich in hydrogen and carbon monoxide for use as a primary feedstock in chemical processes or as a fuel. The invention provides environmentally safe, efficient and versatile processing of natural or synthetic organic materials of single or mixed origin, and of highly variable particle size and shape.

The invention also provides a method and apparatus for generating a char that is a vitreous blend of substantially inert materials that are landfill safe and/or have commercial applications as a vitreous material. Examples of vitreous materials with commercial applications are brick, tile, pigments, filler and ceramics. The invention also provides for separating oversized residual materials from the char from the materials that are to be vitrified.

In particular, the invention provides a single rotating reactor that has two contiguous hearth reaction zones, a first zone that is a drying and volatizing area and a second zone that is a reformation area, where the zones are separated by an internal refractory weir with an aperture that fluidly connects the two reaction zones. In each of the two reaction zones the temperature, pressure, and chemical characteristics of the internal gaseous atmosphere can be selectively controlled to achieve the degree of volatilizing, cracking, dissociation, and/or reforming of vaporous hydrocarbon gases that is required to meet the desired operating objectives. Solid waste organic material is fed into the first zone of the reactor via a conveyor fitted with an air lock. The air lock occludes most of the ambient air, and in particular nitrogen. The rotary reactor has a first zone oxy-fuel burner for heating the waste organic material to a temperature of about 500° C. to about 600° C. The burner employs oxygen that is substantially free of nitrogen. The fuel is typically natural gas, propane, butane, fuel oil, coal dust, or a blend thereof. The first zone oxy-fuel burner provides a flame that substantially is directed above the feed materials so that combustion of the feed material is minimal. By the method of the invention, as new organic feed material enters the reactor, it is quickly heated. The additional organic feed material is retained in the drying and volatilizing zone by the internal weir, and admixed with previously heated residual solid matter in a common bed of matter until the new feed material is completely dried and volatilized. The dried and volatized residual solid matter and the resulting process gasses pass through the refractory weir into the reformation area of the reactor. The reformation area of the reactor has a second zone oxy-fuel burner that also is directed substantially above the bed of matter, and provides sufficient heat, on the order of about 600° C. to about 1000° C., to effect thermal cracking and dissociation of the volatilized organic material to form a synthesis gas rich in hydrogen and carbon monoxide.

At an exit of the rotary reactor there is a gas discharge duct through which exits the gaseous mixture rich in hydrogen, and a discharge port through which exits the ash residue.

Depending on the composition of the feed material, there can be a need to add water, oxygen or even supplemental fuel, and the reactor can have an enrichment injection port in the second zone that enables the stoichiometry of the synthesis gaseous mixture to be shifted toward a gas having a higher heat of combustion, or a higher weight percent of molecular hydrogen. For instance, if it is desired that the gaseous mixture have a higher percentage of hydrogen, then to assure that a greater percentage of the volatile organic compounds are broken down to carbon monoxide and hydrogen, the enrichment injection port can be used to add water. The oxygen in the water oxidizes the carbon forming carbon monoxide and hydrogen. As the reaction is endothermic, it may also be necessary to lower the through put to assure that the temperatures are hot enough to keep the synthesis gas reaction equilibrium shifted toward hydrogen. If a lower level of hydrogen is acceptable, then the enrichment injection port can be used to add fuel and/or lower the temperatures and increase through put. If the waste organic materials is particularly high in carbon content, such as polypropylene or polyethylene, then enrichment injection port can be used to inject pure oxygen to oxidize the carbon to carbon monoxide and hydrogen.

The synthesis gas produced by the apparatus can be purified (i.e., using cyclonic and filter apparatus, activated carbon beds, scrubbers, shift reactors, hydrogen sieving, hydrogen separation, sieves, and other purification apparatus) so that it is suitable for a fuel cell, transportation, chemical, industrial, pharmaceutical, energy, and food industry applications.

Alternatively, the synthesis gas produced by the apparatus can be converted into organic acids and alcohols by a fermentation process. After passing through a gas scrubbing system, the synthesis gas is then passed through a bioreactor, which are usually large fermentation vats where aqueous solutions containing special anaerobic bacteria consume carbon monoxide and hydrogen from the synthesis gas and produce organic alcohols and acids. These products can then be recovered separately as high value added products. Hydrocarbon gases contained in the TRG synthesis gas are not consumed by the bacteria and pass through the fermentation process along with carbon dioxide and nitrogen, as a residual gas. The residual fermentation gas is referred to as FermGas, which has some heating value. FermGas can be used to provide fuel for the TRG reactor or to generate electric power via turbines or collected for use at a later time.

The apparatus is engineered so that it can accept a variety of renewable, organic feedstock materials, and in particular waste streams generated by municipalities, farming, and certain industries. Feedstock for the apparatus is or will be nearby, and continuously generated by the public in the way of garbage. The economics of scale are more than offset by the ready availability of the fuel supply at a cost that is substantially free except for the cost of delivery.

In the method, waste organic material is mechanically metered into the drying and volatizing area of the TRG reactor and quickly heated to a temperature of about 500° C. to about 600° C. by heat transfer methods that, preferably, include at least one volatizing oxy-fuel burner in zone 1. The organic feed material is retained in the drying and volatizing area by the internal weir that substantially restrains the solid materials until they reach a temperature that approaches the upper temperature limit of the first zone and then spills over to the second zone where optimally they heat enough to become mixed with previously heated residual solid matter in a common bed of matter. The dried and volatized residual bed matter and resulting process gases then pass to the second zone of the reactor, where the gases are cracked and dissociated. The flame provided by the oxy-fuel burner in zone 1 and zone 2 gasifies the organic components of the feed material, breaking the hydrocarbons down to small molecules, and then reforms the hydrocarbons into substantially carbon monoxide and hydrogen. The resulting synthesis gas can be used as is, as a fuel, or purified into hydrogen.

### BRIEF DESCRIPTION OF THE DRAWINGS

The foregoing and other objects will become readily apparent by referring to the following detailed description and the appended drawings in which:

FIG. 1 is a schematic cross sectional view of a preferred embodiment of the present invention in which the methods of the invention may be practiced for gasifying organic materials.

FIG. 2 is a schematic cross sectional view of the inter-rotation of the feed material in the rotary reactor having a refractory surface.

FIG. 3 is a schematic view of various cogeneration systems that can be combined with the apparatus.

FIG. 4 is a schematic view of a TRG (Thermal Reduction-Gasification) process, wherein synthesis gas is produced, which can be used as a fuel source to generate electricity and hydrogen.

FIG. 5 is a schematic view of a TRG process, wherein the synthesis gas is converted to organic alcohols and organic acids via fermentation. Non-metabolized gases containing gaseous organic hydrocarbons, typically C1 to C3 gases, can be utilized as a fuel source.

### DETAILED DESCRIPTION OF THE INVENTION

The invention provides a single reactor having two contiguous hearth reaction zones, a first zone, which is a drying and volatizing area and a second zone, which is a reformation area and a hearth melting area. There is a bed retaining radial weir located between, and common to, the two reaction zones. In the illustrated embodiment, the radial weir has an aperture for fluidly connecting the two hearth reaction areas of the reactor. The single reactor of the invention is optimized to generate a synthesis gas having hydrogen from waste organic materials.

A synthesis gas of a desired chemical composition may be produced and reformed within a single reactor, without the need for a downstream secondary reformation or “finishing” reactor. Moreover, by precisely controlling the temperature, pressure and chemical characteristics of the input burner(s), and the gaseous atmosphere in the two reaction areas of the single reactor, the design equilibrium chemical composition of the synthesis gas may be obtained.

Although the invention may be practiced in another type of vessel by one skilled in the art of pyrolysis and gasification, the preferred reactor in which the invention is most easily and preferably practiced is a rotary reactor that revolves around its longitudinal axis and is disposed either horizontally or with a slight incline with respect to its axis of rotation. The feed material is tumbled forward toward the discharge end, even if the rotary reactor is horizontally disposed. The mass flow rate of a bed of heated solid matter through the single reactor is controllable by the rotational speed of the reactor, the height of the weir.

The entire reactor is insulated and refractory lined and can, therefore, be safely and repeatedly heated to internal temperatures up to 1000° C., without sustaining structural damage. The maximum allowable temperature is dependent on the lower fusion temperature of the associated inorganic solids. Exemplary of a rotary reactor is a rotary kiln. A typical rotary kiln suitable for use in the invention has a carbon steel shell lined with about 3 to 4 inches of insulation and about 6 to 9 inches of hot face refractory, sufficient to keep the temperature of the shell exposed to the outside atmosphere at an acceptable level.

The TRG (Thermal Reduction-Gasification) process is designed to gasify social waste and/or natural organic based solid or liquid materials (biomass) containing carbon, hydrocarbon and/or cellulose matter. It is a high temperature, low pressure process that rapidly gasifies and converts such organic feed materials directly into high-grade synthesis gas (a.k.a., syngas). TRG syngas typically contains between 65% to 75% of carbon monoxide and hydrogen, in approximately equal molar amounts, 10% to 20% one and two carbon hydrocarbons, and 12% to 18% carbon dioxide plus nitrogen, on a dry basis. As produced, TRG syngas contains about 7% moisture.

Table 1 gives a break down of typical municipal garbage, which is anticipated to be a major source of feed material fuel for the reactor.

Table 2 gives the composition of a TRG Synthesis Gas based on a generic feedstock of municipal solid waste. A reactor generating 350K scf/hr will generate about 126 million BTU/hr.

Table 3 gives the composition of the ash.

TABLE 1

| | As Received | Dry |
|---|---|---|
| | Wt % | Wt % |
| Overview | | |
| Moisture | 42.3 | 0.0 |
| Volatile Matter | 44.3 | 76.8 |
| Fixed Carbon | 5.6 | 9.7 |
| Ash | 7.8 | 13.5 |
| Elemental Analysis | | |
| Hydrogen | 7.6 | 5 |
| Carbon | 27.2 | 47.3 |
| Nitrogen | 0.8 | 1.4 |
| Oxygen | 56.5 | 32.6 |
| Sulfur | 0.1 | 0.2 |
| Ash | 7.8 | 13.5 |
| Total | 100 | 100 |
| BTU/Lb | 5,310 | 9,180 |

TABLE 2

| | Dry Syngas | Wet Syngas |
|---|---|---|
| Composition | % Vol | % Vol |
| Hydrogen | 36.22 | 33.11 |
| Carbon Monoxide | 35.15 | 32.66 |
| Methane | 8.13 | 7.55 |
| Acetylene | 0.37 | 0.34 |
| Ethylene | 1.82 | 1.69 |
| Ethane | 0.38 | 0.36 |
| Other Hydrocarbons | 0.17 | 0.16 |
| Carbon Dioxide | 17.40 | 16.17 |
| Nitrogen | 0.34 | 0.32 |
| Water Vapor | 0.00 | 7.09 |
| Total | 100 | 100 |
| BTU/scf | 354 | 329 |

TABLE 3

| | Bottom Ash | Combined Ash |
|---|---|---|
| Composition | Wt % | Wt % |
| Silicates | 16.8-20.6 | 13.8-20.5 |
| Lime | 7.1-7.7 | 5.4-8.0 |
| Iron Oxides | 2.1-9.3 | 2.9-7.9 |
| Aluminum Oxides | 4.7-5.6 | 3.3-5.5 |

The reactor of the invention has at least two burners and at least one enrichment injection port for providing the thermal energy and combustion products necessary for the process(es). At least one first zone burner is located in the feed end of the reactor to provide high temperature combustion products and energy for drying and volatizing the organic feed material, and at least one second zone burner is located in the discharge end of the reactor to provide additional high temperature energy and combustion products for heating the solid residual mass, as well as the hydrocarbon-laden vapors and/or fumes entering the reformation area from the drying and volatizing area. While it would be possible to use other types of process burners, the type of burner most preferred by this invention is one that uses pure or near pure oxygen mixed with an appropriate gas, oil, coal dust or mixture to provide the necessary process heat and atmospheric chemistry. Suitable oxy-fuel burners for use in the invention method are available from commercial suppliers known to those skilled in the art. The process burners may be either water-cooled or gas-cooled or cooled by other means known to those skilled in the art. The enrichment injection port is employed to inject pure or near pure oxygen directly into the high temperature atmosphere of the fuel-rich (synthesis gas) reformation area when sufficient atmospheric temperature and combustible in situ gases are present. Occasionally, additional fuel or steam may also be preferably injected. Suitable enrichment injection ports for use in the invention are available from commercial suppliers known to those skilled in the art.

By the method of the invention, the feed material, which preferably has a particle size that may range from about 4 inches down to dust size particles, is metered into the drying and volatizing area of the reactor through atmospheric locking devices that prevent the ingress of free air into, and the egress of hot process gases out of, the vessel. A purge gas such as carbon dioxide or steam can be further employed to prevent the entrance of air. High temperature seals, known to those skilled in the art, are employed throughout the reactor to prevent seepage of air and gases and to maintain a desired pressure inside the reactor. The pressure in the reactor is preferably stabilized at between negative 1.0 and positive 1.0 inches of water gauge by means of a positive displacement pump that exerts a negative pressure on the reactor by pumping out gas, and a balancing positive pressure on the reactor by piping a small portion of clean pressurized gas back into the reactor, resulting in the desired negative to positive pressure balance. Preferably, the pressure is maintained slightly negative, in order to not pressurize the atmospheric seals any more than necessary.

In the drying and volatizing area of the reactor, the process temperature can be adjusted to any desired level by selectively adjusting the input of energy from the first zone volatizing burner 2. Preferably, the feed material 9 is quickly heated to temperatures of about 500° C. to 600° C. for the purpose of completely removing free moisture and vaporizing volatile organic matter from the feed material 9. As stated above, the preferred first zone volatizing burner is an oxy-fuel burner having the ability to operate with injected oxygen gas and fuel gas at ratios ranging from sub- to super-stoichiometry, depending on operating objectives. Preferably, the input burner gas ratios can be varied from 1.75:1 to as high as 10:1. Volatized organic gases emanating from the feed material may also be consumed by partial combustion with super stoichiometric oxygen injected when the flow of burner supplied fuel gas is reduced according to operating objectives.

The first zone burner fires directly into the drying and volatizing area from the feed end of the reactor and is positioned within the reactor so as to avoid direct impingement of the flame with the bed feed material and hearth refractories in order to prevent carbonizing on the kiln walls. Slagging of the residual solid matter can be further prevented by precisely controlling the atmospheric temperature in the drying and volatizing area to prevent reaching the fusion temperature of the solid matter. The products of combustion (carbon dioxide and water vapor) from the first zone burner, and the hydrocarbon-laden gases evolved from the bed of the feed material, flow in a co-current direction with the solid residual matter toward the reformation hearth area.

The evolved process gases and solid residual matter flow from zone 1 into zone 2 by passing over the radial weir, described above, which retains the solid residual bed matter in the drying and volatizing area for a sufficient amount of time to allow substantially complete drying and volatization to occur.

In the reformation area, the hydrocarbon-laden vapors entering from the drying and volatizing area are subjected to controlled temperatures that may be varied between 600° C. and 1000° C., the temperature being selected according to the operating objectives. The high temperature process energy is provided by the second zone burner, which is located in the discharge end of the reactor and fires directly into the reformation reaction area. The burner is positioned within the reactor so as to avoid direct impingement of the flame with the residual solid materials and the hearth refractories in order to prevent carbonizing on the reactor walls. The solid materials are prevented from fusing by precisely controlling the temperature in the reformation area to keep it below the temperature for incipient fusion.

The preferred method of operation is to use the second zone burner to preheat the rotary reactor and process gas handling systems before feed material is introduced into the reactor. After the start of the raw feed into the reactor, and after establishing proper operating temperatures and reaching desired chemical equilibrium in both the drying and volatizing area and the reformation area, the second zone burner firing rate may be incrementally reduced to the low fire level, while systemically replacing the process energy needs by direct injection of oxygen into the reformation area via the enrichment injection port.

The second zone burner and/or the enrichment injection port fires its products of combustion in a counter-current direction relative to the flow of the process gases, and the dust and solids from the drying and volatizing area of the reactor in order to thoroughly mix the products of combustion from the burner with the process gases. Thus, most of the process (hydrocarbon-laden) vapors become quickly and intensively mixed with very high temperature oxidizing agents (CO₂ and H₂O) from the second zone burner. The organic vapors are rapidly cracked, disassociated, and/or reformed into a synthesis gas that is rich in hydrogen and carbon monoxide, and little or no condensable hydrocarbon vapors remain in the gaseous product.

Another important aspect of the methods and apparatus of the invention is that most, if not all, of the solid fixed carbon remaining in the residual bed matter passing from the drying and volatizing area into the reformation area of the reactor is converted to synthesis gas. By the process, the solid fixed carbon in the reformation area is sufficiently elevated in temperature, in the presence of water vapor (from either the inherent humidity in the reactor or water vapor formed as a combustion product CH₄+2O₂→CO₂+2H₂O by the second zone burner), to be subjected to water gas reactions to form carbon monoxide and hydrogen gas, according to the reactions (1) and (2).

C+H₂O →CO+H₂(ΔH=−31,380 cal./mole C) (1)

or

C+CO₂→2CO(ΔH=+41,220 cal./mole C) (2)

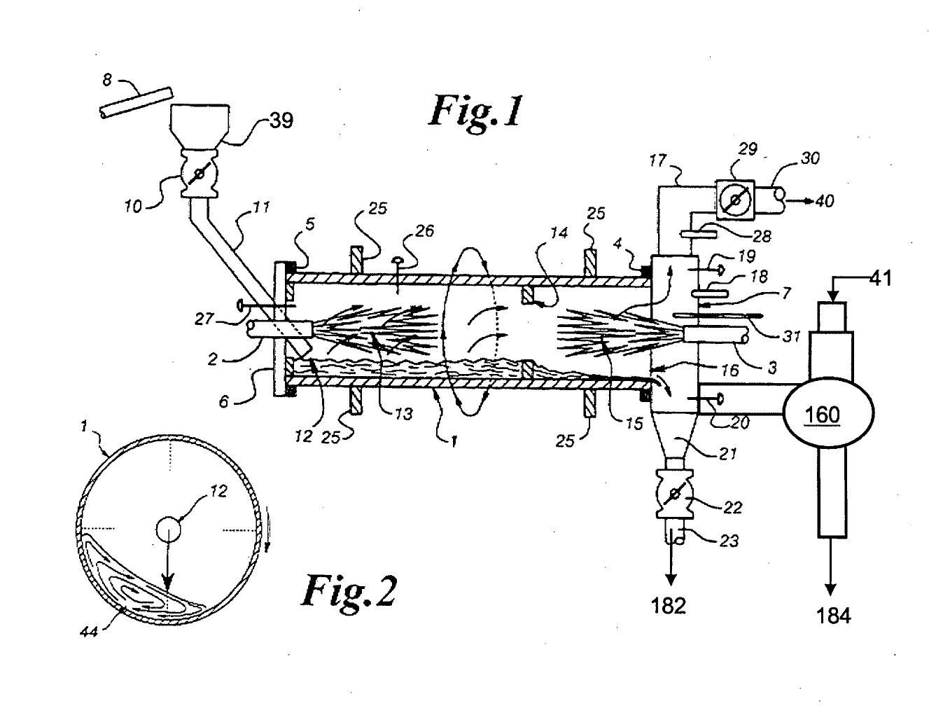

The TRG reactor in which the invention is practiced as illustrated in FIG. 1. Prior to starting the feeding of raw materials 9 into the process, the reactor 1 is purged of air to provide an air free gas atmosphere, and also pre-heated, as described above, to process temperatures in both zones of the reactor. The first zone burner and/or the second zone burner may be fired under stoichiometric or sub-stoichiometric conditions to provide the energy and atmospheric gases needed for pre-heating the hearth areas to a temperature of about 650° C. to 750° C. and to purge air out of the reactor. Purging of the reactor and downstream gas processing system may also be accomplished by circulation of the waste combustion gases (CO₂+H₂O) from the first zone burner and/or the second zone burner. The recycled gases, which have been scrubbed and cooled to near atmospheric temperature, provide mass and volume, but not heat energy. The waste combustion gases are drafted from the reactor through the entire process system, including downstream gas handling and cleaning systems, by induction draft, thus purging the entire system of air.

Referring to FIG. 1, which represents a preferred embodiment of the present invention, feed material 9 of partial or total organic composition is metered into a holding hopper 39 by a metering and conveying device 8. The feed material 9 is mechanically fed into a pre-heated, free oxygen-purged reactor 1 through an atmosphere locking rotary or double dump valve 10 and flows by gravity through the raw feed conduit 11 into the entrance area 12 of the drying and volatizing area 13 of the reactor 1. Depending on the nature of the selected feed material for the process, it may be necessary to employ feeders that have the ability to feed non-free flowing and/or sticky materials by mechanical means which are commercially available and well known to those practiced in the art.

By the rotating action of the reactor 1, discussed further below, the feed material 9 is quickly mixed with previously heated bed of residual solid matter that resides in the drying and volatizing area 13 of the reactor 1. The residual matter is composed of particles and granules of inorganic matter and carbon char. Heat is quickly exchanged from the hot bed, apparatus walls, and gases in the atmosphere of the drying and volatizing area 13, into the new organic feed material. In the preferred embodiment, a first zone burner 2 is employed directly inside the entrance area to offset the endothermic exchange of heat between the new material and the previously heated bed of residual solid matter. The products of combustion from the first zone burner 2, plus the gases evolved from the new organic feed material flow in a co-current direction with the residual solid matter.

In a preferred embodiment, the reactor 1 is a rotary reactor fabricated from carbon steel and is lined inside by fire brick or similar quality castable refractories that are able to withstand the potentially damaging effects of both high temperature and/or chemical alteration. The supporting and rotating devices 25 of the reactor are of standard mechanical design and may be supplied by any number of commercial manufacturers of rotary kilns. The longitudinal axis of the reactor 1 may is substantially horizontal. The principal function of the reactor is to contain, mix and convey the material mass and generated gases from the feed end to the discharge end of the apparatus, while maintaining a protected high temperature atmosphere. Atmosphere seals 4, 5 are located between the rotating reactor 1 and the feed end hood 6, and the discharge end hood 7 fixed structures. These seals allow slippage between the rotating reactor and the non-rotating fixed structures without allowing ingress of atmospheric air into the process, or egress of hot process gases out of the processing apparatus into the plant work area. Such seals are commonly known and can be supplied by commercial manufacturers of rotary kilns.

At least one protected thermocouple 26, located at any convenient point along the shell of the reactor 1 and extending through the shell and fire brick into the inside atmosphere of the drying and volatizing area 13 is provided to allow monitoring of the atmospheric temperature in that area. Additionally, a control thermocouple 27 is located in the feed end hood 6 for the purpose of monitoring the temperature of the atmosphere at the entrance of the drying and volatizing area 13 and for controlling the first zone burner 2 by means of feed-back electronic signals to burner controlling devices (not shown).

Once the feed material 9 is introduced into the drying and volatizing area 13 of the reactor 1, the material is immediately subjected to the high temperature (500° C. to 600° C.) of both the previously dried and volatized residual bed material and the hot process gases in that area. The temperature within the drying and volatizing area 13 is maintained by very high temperature products of combustion generated by the first zone burner 2, which is programmed to automatically control the burner combustibles at a level sufficient to maintain the desired temperature in the drying and volatizing area 13. The first zone burner 2 may be of standard commercial design and may utilize any suitable source of fuel (including organic vapors residing within the atmosphere of the drying and volatizing area of the reactor) for direct combustion with either pure oxygen, or a suitable blend of oxygen and air (see below), as needed, to deliver the selected level of energy into the drying and volatizing area. Combustion may alternatively take place by blowing compressed air through the burner into the reactor. However, this method is not preferred because the high nitrogen content in atmospheric air may greatly increase the gas volume and contaminate the synthesis gas 40 with inert nitrogen gas. Combustion may also alternatively take place with a blend of natural air and pure oxygen and may achieve a lower process cost; however, as above, the added nitrogen from the air would have to be taken into consideration in design of the plant. The preferred combustion method of the invention employs pure oxygen, primarily to exclude the contaminating effect of nitrogen that would be introduced with air.

In the drying and volatizing area 13 of the reactor 1, the feed material is quickly heated above the boiling point of water and the bed feed material is freed of all non-combined water. Upon reaching the dry state, the feed material continues to be elevated in temperature to a level between 500° C. and 600° C., while remaining in the drying and volatizing area 13. Volatile matter contained in the feed material begins to volatize at about 120° C. and, by the time the solid mass of the feed material reaches a temperature of about 350° C., most, if not all, of the volatile matter contained in the original feed material is liberated to the vapor state. Some tar-forming hydrocarbons are more refractory and may not complete volatizing until the temperature of the solid mass exceeds about 450° C.

Depending on the moisture content and type of feed material selected for the process, the first zone burner 2 has the capacity to provide between 2.0 and 4.0 million Btu per hour per ton of feed material into the drying and volatizing area 13. For example, different types and densities of feed materials require more or less heat energy in order to reach the processing temperature, i.e., 500° C. to 600° C. The amount of ash in the feed material may also influence the level of burner energy required.

By the time the solid mass remaining as residue of the original feed material reaches the bed retaining refractory weir 14 in reactor 1, the temperature of the mass will reach a temperature between 500° C. and 600° C. and most, if not all, carbon remaining in the solid mass will be fixed.

The weir 14 is disposed substantially perpendicular to the longitudinal axis of the reactor 1 and is positioned along the longitudinal axis in such a location as to provide about 30 minutes to 60 minutes of retention time, depending on the speed of rotation of the reactor 1 and the rate of feed of raw material into the reactor. The depth of residual solid matter retained in the drying and volatizing area 13 is determined by the height (or aperture) of the weir 14. Generally, the aperture of the weir is set sufficiently high to allow a working bed depth in the rotary reactor 1 equal to about 12% to about 15% of the total available volume in the drying and volatizing area 13. This bed depth is an important factor in causing inter-rotation of the bed to provide uniform mixing of the bed materials and for achieving optimum processing capacity. Inter-rotation of the bed material 44 on axis 12, illustrated in FIG. 2, vastly increases the potential for heat transfer into the center of the rotating bed 42. Thus, fresh organic feed materials entering the drying and volatizing area 13 become quickly intermixed with hot residual matter due to the rotation action of both the material bed and the refractory hearth of the reactor 1.

The retention time of residual solid matter in the drying and volatizing area 13 should normally be between thirty and sixty minutes, depending on the relative content of hydrogen compared to carbon in the feed material. The residual solid mass passing from the drying and volatizing area 13, over the bed retaining weir 14, and into the reformation area 15 of the reactor 1, is further mixed and heated in the hearth 16 of the reformation area 15. Additional high temperature oxidizing agents (CO₂, H₂O and O₂) are injected into the reformation area 15 by either the second zone burner 3, or the water- or gas-cooled enrichment injection port 31 that is located in the discharge end of the reactor 1. The products of combustion of the second zone burner 3 are fired in a counter-current direction relative to the flow of both the gases and the solids from the drying and volatizing area 13 of the apparatus.

The hydrocarbon-laden gases and/or fumes entering the reformation area 15 are largely composed of condensable complex hydrocarbon chains. At the high temperatures present in the reformation area 15, free oxygen may be present either due to an excess of oxygen from the second zone burner oxygen/fuel mixture, or from the injection of free oxygen directly into the reaction area through the enrichment injection port 31. The free oxygen reacts first with the hydrogen and the lightest available hydrocarbon, which is usually methane, to form carbon dioxide and water vapor in an exothermic reaction. Under the high temperature conditions of the flame front in the reformation area 15, both carbon dioxide and water vapor act as oxidizers that secondarily react endothermically with complex hydrocarbon-laden vapors and/or fumes to produce synthesis gas 40 and less complex hydrocarbon gases. The less complex hydrocarbon gases are further oxidized by oxygen, carbon dioxide and/or water vapor to produce more carbon monoxide, hydrogen and carbon dioxide gases. The higher the temperature, the faster the partial oxidation reactions occur, and the more of the complex hydrocarbons are converted to carbon monoxide, hydrogen and carbon dioxide gases. Thus, by selectively controlling the temperature and gaseous atmospheric environment of the reformation area 15, the quality of the resulting non-condensable synthesis gas 40 can be produced having higher heating values (HHV) of between about 275 and 402 Btu/standard cubic foot (Btu/cu ft). Because the evolved process gases are reformed into synthesis gas 40 within a single reaction vessel, there is no requirement for a secondary reactor downstream of the primary reactor 1.

By the method of the invention, with the available atmospheric oxidants described above and the process temperature in the reformation area being maintained between 500° C. and 600° C., the resulting process gas comprises about 15% to about 20% by volume each of carbon monoxide and hydrogen, about 20% to about 25% hydrocarbon gases containing one to two carbon molecules, and about 15% to about 20% hydrocarbon gases containing more than two carbon molecules.

If it is desirable to obtain a higher synthesis gas 40 with a higher content of hydrogen, the temperature of the gases and the solid residual matter may easily be increased as high as 1000° C. for the purpose of reforming part or most of the hydrocarbon vapors and carbon soot (fume) and much of the carbon-rich solid residue, into synthesis gas 40 (hydrogen and carbon monoxide). The second zone burner 3 and/or the enrichment injection port 31 can be manipulated and controlled by the nature of the ratio and quantity of oxygen-to-fuel selected for the burner or enrichment injection port to provide the high temperature energy and gaseous oxidants required to achieve an optimum level of composition equilibrium to meet operating objectives.

The residual mass remains in the reformation area 15 for only a few minutes before passing out of the reformation area of the reactor 1 through a liquid-solid discharge port to a solids collecting chute 21 and is metered out through a solids flow control device 22 that also serves as an atmospheric seal for the process. The flow control device may be any suitable type of rotary or double dump valve that is available from numerous commercial sources. The temperature of the exiting residual mass can be measured and monitored by a thermocouple 20 and the temperature of the second zone burner or enrichment injection port adjusted accordingly. The hot solids evacuation duct 23 then conveys the residual mass of material via a connecting conduit to a cooling device. The methods and type of equipment needed to receive and cool the hot residual mass, which may be a latent vitreous mixture, as well as to further process the material by conveying, screening, bagging, briquetting, storing or otherwise handling the cooled mass as a final product, are well known to those practiced in the art and the equipment is readily available from commercial suppliers.

As shown in FIG. 4, the residual mass of material can be separated into oversized residual material 182 and vitrifiable material 184. The vitrifiable material 184 moves into the vitrifier 160. Optionally, the vitrifiable material 184 can further be comprised of particulate and dust 41 collected by scrubbing apparatus 162 from the synthesis gas 40 or bottom ash from incineration of municipal solid waste generated at a Waste to Energy Plant. If required, silicates, clays, alumina and other vitreous materials can be added to the vitrifier 160 to increase the value of the resulting glass 184, or to augment the process.

As shown in FIG. 1, the thermocouple 19 is located in the discharge hood 7 near the entrance to the discharge duct 17 for the purpose of monitoring the temperature of the exiting gaseous mass and for transmitting electronic control signals to second zone burner 3 and/or enrichment injection port 31 metering central equipment (not shown). Thus, the second zone burner and/or the enrichment injection port can be programmed to automatically adjust as needed to maintain the temperature at a prescribed level in the reformation area. Typically, when the temperature of the reformation area is maintained at about 650° C. to 750° C., the resulting synthesis gas comprises from about 30% to about 35% by volume of each of carbon monoxide and hydrogen gas, about 3.5% by volume of gases with a molecular structure having two carbon atoms, and about 1.5% by volume of gases with molecular structure having more than two carbon atoms. When a higher level of reformation is required, it is necessary to increase the reformation temperature from the 650° C. to 750° C. level to between 750° C. and 1000° C. This increase in temperature requires additional energy input in the reformation area 15 supplied by either (or both) the second zone burner 3 or the enrichment injection port 31. The additional energy input is needed to raise both the residual solid matter and the process gas stream to the desired temperature. Under typical operating conditions, between one and three million Btu/hour additional energy input would be required per ton of feed, depending on the characteristics of the feed material. The resulting synthesis gas comprises a higher percentage by volume (about 35% to about 40%) of each of carbon monoxide and hydrogen gas; however, the volume of gases containing two carbon molecules is reduced to less than 1%, while the volume of gases containing more than two carbon molecules is reduced to less than one half percent. The heating value (HHV) of this gas is lowered to about 275 Btu/cu ft due to the reduction of hydrocarbon gas and increase in carbon monoxide and hydrogen gases. Although this gas could be used as a fuel for combustion purposes, its higher level of carbon monoxide and hydrogen makes the gas better suited for use as a feedstock for the commercial production of organic chemicals, and in the specific application for recovering and increasing the yield of hydrogen.

The operating pressure in the reactor 1 and in the discharge hood 7 is controlled by a variable speed induction draft fan or blower (not shown) that is located downstream of a process pressure trim valve 29. A further embodiment of the pressure control includes the recycling of a controlled portion of the pressurized cooled and cleaned product gas back into the discharge hood through the recycle gas pipe 28. The recycled gas system also serves to stabilize the inert gaseous temperature, pressure, and atmosphere throughout the reactor, and the cooling, condensing, and gas cleaning systems during the period of time that the systems are being pre-heated and prior to starting the feeding of feed material into the process. The product gas exiting through the process pressure trim valve is ducted 30 to and through several stages of gas cooling, condensing, and cleaning equipment that is well known to those practiced in the art and readily available from commercial suppliers of such equipment.

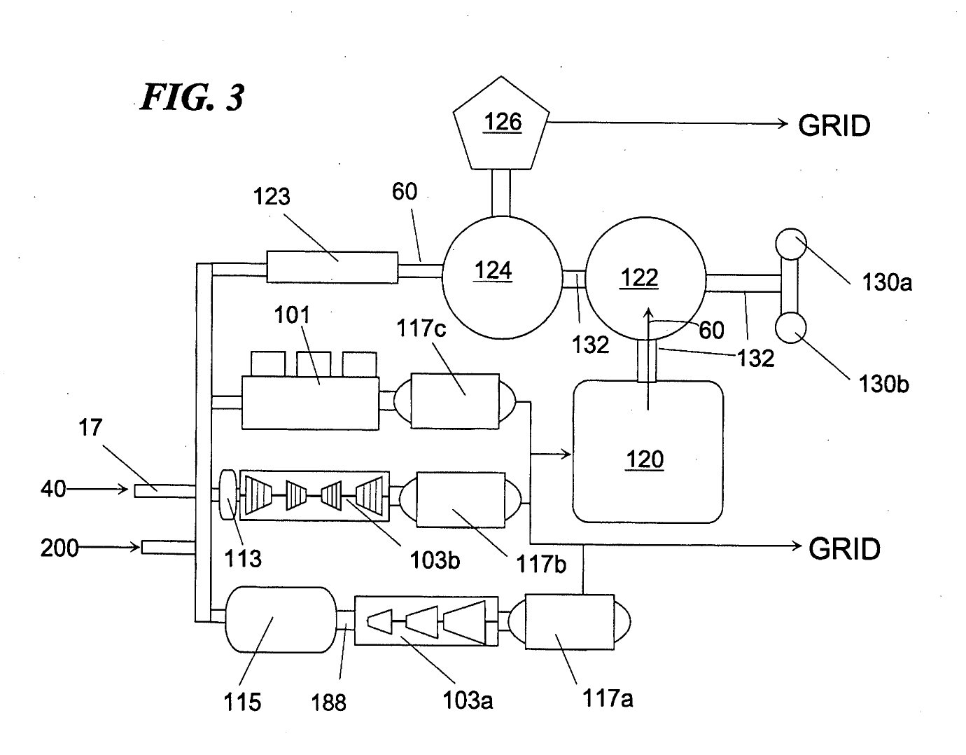

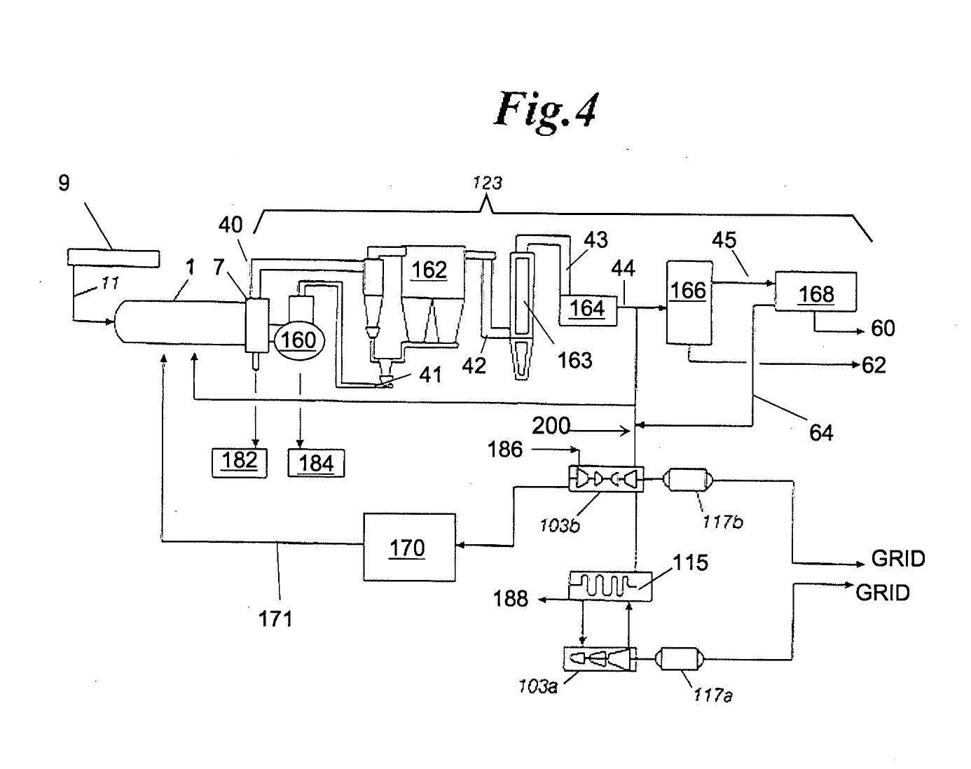

The synthesis gas 40 exiting through the discharge duct 17 is optimized for the intended application. As shown in FIG. 3, the apparatus can be optimized to generate a fuel for an internal combustion engine 101 that has been modified to run off synthesis gas. As illustrated, the internal combustion engine 101 powers a third electrical generator 117c, which via an electrolysis cell 120 produces pure hydrogen 60 from water. The hydrogen 60 is distributed via pipes 132 to the primary tank 122, the distribution points 130a and 130b, and the fuel cell storage tank 124 for use in the fuel cell 126. The fuel cell 126 can generate electricity for the electrical power grid and for the apparatus 1. Alternatively, synthesis gas 40 can be used to power and a gas turbine 103b having a first electrical generator 117b. Turbines require input pressure in excess of 200 psi, and the synthesis gas 40 will need additional pressure and could be augmented by additional combustion gases. Typically, these would be provided by additional oxy-fuel burners feeding a combustion chamber 113 for the turbine 103b. In another embodiment, the steam turbine 103a drives second electric generator 103a. The synthesis gas 40 is used to power a boiler 115, which generates the steam for the turbine 103a. The turbines 103a and 103b can be augmented by heat generated by conventional fuels, such as LPG, NG or fuel oil when and where required, and these fuels are generally shown as 200. Hydrogen 60 generated by electrolysis is very pure, and is suitable for fuel cells. The hydrogen can be stored on site in primary tank 122 to be dispensed to vehicles by terminals 130a and 130b, or stored in the fuel cell storage tank 124 for use in the fuel cell 126. The hydrogen can be stored in low pressure storage tanks, or compressed to be delivered to other outlets proximal to the generation site. The dispenser terminals can fuel vehicles, tanker trucks, railroad tankers, portable cylinders, and cryogenic containers. Alternatively, the stored hydrogen can be used onsite via the fuel cell 126 to provide another source of electricity during peak demand for electricity. In another variation, as shown in FIG. 3, the synthesis gas 40 can be stripped and scrubbed 123 removing all components other than hydrogen 60, and the hydrogen can be stored in the fuel cell storage tank 124.

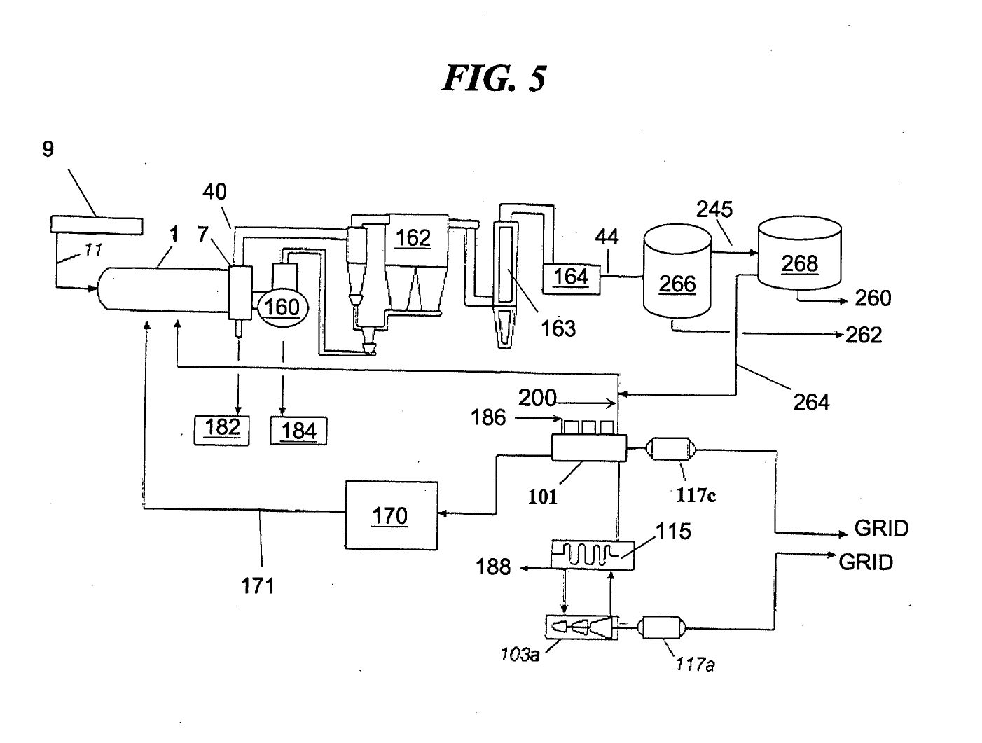

The process for stripping, scrubbing, and purifying is schematically represented in FIG. 3 as 123. FIG. 4 has the details of the process and apparatus. The bracket 123 generally designates the components consisting of particulate removal apparatus 162, gas cleanup apparatus 163 and 164, shift reactor 166 and hydrogen separation unit 168. The particulate removal apparatus 162 largely ash, dust and some condensable metals 41. These particulates are returned to the vitrifier 160. The shift reactor typically utilizes steam to convert carbon monoxide to carbon dioxide and additional hydrogen. As shown in the diagram, the purified synthesis gas 44, prior to shift reactor, can be diverted to gas turbine 103b or the illustrated internal combustion engine 101 in FIG. 5. The synthesis gas is burned with the addition of air 186. The synthesis gas can be enriched with conventional fuels 200 and hydrocarbon homologs 64 concentrated in the hydrogen separation unit 168. Exhaust gases exiting the internal combustion engine 101 as shown in FIG. 5, or the gas turbine 103b as shown in FIG. 4, can be used in a heat recovery steam generation unit 115 (i.e. a boiler) to generate steam 188 for power the steam turbine 103a. The internal combustion engine 101 drives generator 117c, and steam turbine 103a drives and generator 117a, where each can generate electricity for the power grid, or be used by the TRG system, for instance to generate hydrogen and power motors. As illustrated, internal combustion engine 101 (or gas turbine 103b, which is not illustrated) is used to compress air 186 piped to an air separator 170. The air separator generates substantially pure oxygen 171, splitting off the nitrogen. The reactor's oxy-fuel burners use the oxygen 171.

FIG. 5 is a schematic view of a TRG process, wherein the synthesis gas is converted to organic alcohols and organic acids via fermentation. Non-metabolized gases containing gaseous organic hydrocarbons, typically C1 to C3 gases, can be utilized as a fuel source. As shown in FIG. 5, scrubbed synthesis gas 44 can be fermented using various strains of bacteria, such as Clostridium, in a bioreactor 266 to produce organic alcohols 262. Reaction 3 illustrates how carbon monoxide can be combined with water to produce ethanol 262, and reaction 4 illustrates how hydrogen can be combined with carbon dioxide to produce ethanol 262. Other alcohols homologs, such as methanol and butanol, have been reported. In the same bioreactor 266, or preferably in a second bioreactor 268, additional fermentation can produce value added organic acids, such as acetic acid 260.

6CO+3H₂O→CH₃CH₂OH+4CO₂ Rx3

or

6H₂+2CO₂→CH₃CH₂OH+3H₂O Rx4

Reaction 5 illustrates how carbon monoxide can be combined with water to produce acetic acid 260, and reaction 4 illustrates how hydrogen can be combined with carbon dioxide to produce acetic acid 260. Other acid homologs, such as butyric acid, have been reported.

4CO+2H₂O →CH₃COOH+2CO₂ Rx5

or

2H₂+4CO₂→CH₃COOH+2H₂O Rx6

The synthesis gas 44 produced by the invented TRG reactor apparatus contains some gaseous compounds, such as methane, propane, and butane, which are not metabolized by the bioreactor. These gases, along with carbon dioxide and small amounts of molecular nitrogen, constitute a biofuel gas 264 known as FermGas. In the invention, the FermGas 264 is used as a fuel for the reactor 1 or a gas turbine 103b. The fuel content can be augmented with conventional fuels, such as LPG, NG, butane, fuel oil and coal dust. These fuels are generally shown as 200. Additionally, the FermGas 264 could be augmented with synthesis gas 44, as shown in FIG. 4.

The descriptions above and the accompanying drawings should be interpreted in the illustrative and not the limited sense. While the invention has been disclosed in connection with the preferred embodiment or embodiments thereof, it should be understood that there may be other embodiments which fall within the scope of the invention as defined by the following claims. Where a claim is expressed as a means or step for performing a specified function, it is intended that such claim be construed to cover the corresponding structure, material, or acts described in the specification and equivalents thereof, including both structural equivalents and equivalent structures.

What is claimed is:

1. An apparatus for generating synthesis gas from waste organic material, said apparatus comprising:
a rotary reactor having a first zone which is a drying and volatilizing hearth reaction area, and a second zone which is a reformation hearth reaction and pyrolysis area, where the zones are separated by a weir that substantially restrains feedstock waste organic material that is fed into the reactor until the waste organic material is fully dried and at least a portion of the organic material is volatilized;
a solid waste organic material conveyor with an air lock that feeds the waste organic material to the first zone of the rotary reactor;
a first zone oxy-fuel burner having a flame, said first zone oxy-fuel burner for heating the waste organic material to a temperature of about 500° C. to about 600° C., wherein the volatized organic material, in contact with the first zone burner flame, is thermally cracked and partially oxidized;
a second zone oxy-fuel burner having a flame, said second zone oxy-fuel burner for heating the dried waste organic material to a temperature of about 600° C. to about 1000° C., wherein the dried waste organic material in the second zone is heated to a char that is a residual mass, and thereby producing additional volatilized organic material, where said volatilized organic material, in contact with the second zone burner flame, is thermally cracked, oxidized, and reformed, therein forming a synthesis gas that is rich in gaseous hydrogen and carbon monoxide;
a gas discharge duct through which exits the synthesis gas;
a solid discharge collection chute through which exits the residual mass; and
an enrichment injection port in the second zone for adjusting the composition of the synthesis gas.

2. The apparatus according to claim 1, wherein the solid discharge collection chute separates the residual mass into oversize material and vitrifiable material.

3. The apparatus according to claim 2, wherein the vitrifiable material is processed in a vitrifier to a glass-like material.

4. The apparatus according to claim 3, wherein additives, which increase the value or augment the vitrification process can be added to the vitrifier.

5. The apparatus according to claim 1, wherein the apparatus is further comprised of components for purifying the synthesis gas, where the components are selected from particulate removal apparatus, and gas cleanup apparatus.

6. The apparatus according to claim 5, wherein particulates collected by the particulate removal apparatus are recycled to a vitrifier.

7. The apparatus according to claim 5, wherein the apparatus is further comprised of components for generating hydrogen, where the components for generating hydrogen are comprised of shift reactor apparatus and hydrogen separation apparatus.

8. The apparatus according to claim 5, wherein the apparatus is further comprised of a gas turbine driving a first electric generator, where said gas turbine is powered by burning synthesis gas.

9. The apparatus according to claim 8, wherein the apparatus is further comprised of a heat recovery steam generator, which captures the hot exhaust gases exiting the gas turbine to generate steam.

10. The apparatus according to claim 9, wherein the apparatus is further comprised of a steam turbine and a second electric generator, where said steam turbine is powered by steam generated by heat recovery steam generator.

11. The apparatus according to claims 8 and 10, wherein the first and second electric generator provide electricity to the power grid, or to an electrolysis cell that generates pure hydrogen or to provide electrical energy to the apparatus utilizing motors or heater, or any combination thereof.

12. The apparatus according to claim 1 is further comprised of an air separation apparatus that provides oxygen to the oxy-fuel burners.

13. The apparatus according to claim 1 is further comprised of an internal combustion motor modified to burn synthesis gas.

14. The apparatus according to claim 13, wherein said internal combustion motor drives a third generator.

15. The apparatus according to claim 1 is further comprised of a gas turbine modified to burn synthesis gas.

16. The apparatus according to claim 15, wherein said gas turbine drives a second generator.

17. The apparatus according to claim 1 is further comprised of a steam turbine having a boiler, wherein the boiler burns synthesis gas.

18. The apparatus according to claim 17, wherein said gas turbine drives a second generator.

19. The apparatus according to any of claims 14, 16 and 18 which is further comprised of an electrolysis cell that generates hydrogen of purity suitable for use in a PEM fuel cell.

20. The apparatus according to claims 19 that is further comprised of at least one hydrogen storage tank.

21. The apparatus according to any of claims 19 and 20 that is further comprised of at least one hydrogen dispenser terminal for vehicles, tanker trucks, railroad tankers, portable cylinders, and cryogenic containers.

22. The apparatus according to any of claims 19 and 20 that is further comprised of at least one hydrogen fuel cell for generating electricity.

23. The apparatus according to claim 1 where the fuel use by the oxy-fuel burner is selected from the group consisting of natural gas, propane, butane, fuel oil, and coal dust.

24. The apparatus according to any of claims 8, 9, 13, 15 and 17 where the synthesis gas is augmented with a fuel selected from the group consisting of natural gas, propane, butane, fuel oil, and coal dust.

25. The apparatus according to claim 5, wherein the apparatus is further comprised of a bioreactor, wherein through fermentation the carbon monoxide and hydrogen comprising the synthesis gas are converted into alcohols.

26. The apparatus as claimed in claim 25, wherein the alcohol is substantially ethanol.

27. The apparatus according to claim 5, wherein the apparatus is further comprised of a bioreactor, wherein through fermentation the carbon monoxide and hydrogen are converted into acids.

28. The apparatus as claimed in claim 27, wherein the acid is substantially acetic acid.

29. The apparatus as claimed in any of claims 25 and 27, wherein nonmetabolized gases produced by the bioreactor constitute a biofuel gas, known as FemGas.

30. The apparatus as claimed in claim 29, wherein the FemGas is used as a burner fuel for the oxy-fuel burners of the rotary reactor, or in boiler for a turbine, or in a motor, such as a gas turbine.

31. The apparatus as claimed in claim 30, wherein the FemGas is enriched with a conventional fuel selected from the group consisting of LPG, NG, butane, fuel oil or coal dust.

32. A cogeneration apparatus, said cogeneration apparatus comprising:
an TRG apparatus for generating synthesis gas from waste organic material, said apparatus comprising:
a rotary reactor having a first zone which is a drying and volatilizing hearth reaction area, and a second zone which is a reformation hearth reaction, where the zones are separated by a weir that substantially restrains the waste organic material that is fed into the reactor until the material is fully dried and at least a portion of the organic material is volatilized;
a solid waste organic material conveyor with an air lock that feeds the waste organic material to the first zone of the rotary reactor;
a first zone oxy-fuel burner having a flame, said first zone oxy-fuel burner for heating the waste organic material to a temperature of about 500° C. to about 600° C., wherein the volatized organic material, in contact with the first zone burner flame, is thermally cracked and partially oxidized;
a second zone oxy-fuel burner having a flame, said second zone oxy-fuel burner for heating the dried waste organic material to a temperature of about 600° C. to about 1000° C., wherein the dried waste organic material in the second zone is heated to a char that is a residual mass, and thereby producing additional volatilized organic material, where said volatilized organic material, in contact with the second zone burner flame, is thermally cracked, oxidized, and reformed, therein forming a synthesis gas that is rich in gaseous hydrogen and carbon monoxide;
a gas discharge duct through which exits the synthesis gas;
a solid discharge collection chute through which exits the residual mass; and
an enrichment injection port in the second zone for adjusting the synthesis gas to have a desired composition;
an engine selected from the group consisting of an internal combustion engine, a gas turbine and a steam turbine, where the motor burns synthesis gas generated by the TRG apparatus; and
a generator, wherein said generator, which is driven by the motor, produces electricity.

33. The cogeneration apparatus according to claim 32 is further comprised of an electrolysis cell, wherein said electricity generates hydrogen.

34. The cogeneration apparatus according to claim 33 is further comprised of a fuel cell.

35. A hydrogen generation apparatus, said hydrogen apparatus comprising:
a TRG apparatus for generating synthesis gas from waste organic material, said apparatus comprising:
a rotary reactor having a first zone which is a drying and volatilizing hearth reaction area, and a second zone which is a reformation hearth reaction, where the zones are separated by a weir that substantially restrains the waste organic material that is fed into the reactor until the material is fully dried and at least a portion of the organic material is volatilized;
a solid waste organic material conveyor with an air lock that feeds the waste organic material to the first zone of the rotary reactor;
a first zone oxy-fuel burner having a flame, said first zone oxy-fuel burner for heating the waste organic material to a temperature of about 500° C. to about 600° C., wherein the volatized organic material, in contact with the first zone burner flame, is thermally cracked and partially oxidized;
a second zone oxy-fuel burner having a flame, said second zone oxy-fuel burner for heating the dried waste organic material to a temperature of about 600° C. to about 1000° C., wherein the dried waste organic material in the second zone is heated to a char that is a residual mass, and thereby producing additional volatilized organic material, where said volatilized organic material, in contact with the second zone burner flame, is thermally cracked, oxidized, and reformed, therein forming a synthesis gas that is rich in gaseous hydrogen and carbon monoxide;
a gas discharge duct through which exits the synthesis gas;
a solid discharge collection chute through which exits the residual mass; and
an enrichment injection port in the second zone for adjusting the composition of the synthesis gas;
purification components for the composition of the synthesis gas, where the purification components are selected from particulate removal apparatus, and gas cleanup apparatus; and
generation components for generating hydrogen, where the generation components are comprised of shift reactor apparatus and hydrogen separation apparatus.

36. The hydrogen generation apparatus according to claim 35 is further comprised of electricity generating components, wherein said electricity generating components use a portion of the synthesis gas to power motors which drive electrical generators.
